(19) United States
(12) Patent Application Publication
(10) Pub. No.: US 2026/0053391 A1
(43) Pub. Date: Feb. 26, 2026

(54) BODY POSTURE MEASUREMENT SYSTEMS AND METHODS

(71) Applicant: WAKE FOREST UNIVERSITY HEALTH SCIENCES, Winston-Salem, NC (US)

(72) Inventors: Tadhg O'Gara, Winston-Salem, NC (US); Kerry Daneison, Winston-Salem, NC (US)

(21) Appl. No.: 19/104,539

(22) PCT Filed: Aug. 16, 2023
(86) PCT No.: PCT/US2023/030354

§ 371 Date: Feb. 18, 2025

Related U.S. Application Data
(60) Provisional application No. 63/398,940, filed on Aug. 18, 2022.

(51) Int. Cl. A61B 5/11; A61B 5/00; A61B 5/01; A61B 5/0205; A61B 5/021; A61B 5/0255; A61B 5/05; A61B 5/145

(52) U.S. Cl. CPC A61B 5/1126; A61B 5/0024; A61B 5/02055; A61B 5/05; A61B 5/1112; A61B 5/6804; A61B 5/01; A61B 5/02141; A61B 5/0255; A61B 5/14542; A61B 2560/0214; A61B 2560/0223; A61B 2560/0242; A61B 2560/0462; A61B 2562/0219; A61B 2562/0223; A61B 2562/0247; A61B 2562/0271; A61B 2562/227

(57) ABSTRACT

The present disclosure relates to posture-related devices and methods including articles of manufacture of a garment with sensors for monitoring and measuring posture of a person.

### CROSS REFERENCE TO RELATED APPLICATION

This application claims priority to and benefit of U.S. provisional patent application Ser. No. 63/398,940 filed Aug. 18, 2022, which is fully incorporated by reference and made a part hereof.

### TECHNICAL FIELD

The present disclosure relates to devices, systems and methods related to monitoring, measuring, and treating a person's posture. In particular, described herein are garments with sensors that are used to monitor and measure body posture.

### BACKGROUND

In some instances, disease and/or injuries to the spinal column or vertebrae are influenced by the posture of a person. For example, scoliosis, spinal arthritis and other conditions may be caused by the poor posture of a person. Such changes may occur rapidly or slowly over time. The condition may progress rapidly between visits to a health care specialist, or there may be little to no change between visits. Monitoring posture and any changes in posture as a person continues their normal day-to-day life on a continuous basis has heretofore not been possible. Therefore, what is needed are devices, systems and methods of continual monitoring of a person's posture that are not overly limiting of the activities of the person.

Recent medical literature suggests that idiopathic scoliosis and low back pain are caused by lack of lumbar lordosis in posture. A mild scoliosis diagnosis will be given with a curvature as minor as 10 degrees (Cobb angle). Postural changes are fairly minimal and often hard for patients and their caregivers to spot at this stage. However, scoliosis is progressive, meaning the condition's natural path is to worsen. The earlier scoliosis is caught and treatment initiated, the less likely a patient's condition will progress to the point where surgery is recommended. Posture changes during this stage are subtle. Knowing what to look for is important and can make the difference between reaching a diagnosis during the early stages when the condition is at its most treatable, or having to tackle treatment when the condition has already progressed significantly.

The present disclosure is directed to overcoming the above and other challenges. While several examples of challenges that arise in a posture-monitoring sensors, attachment devices, and treatment of conditions, other problems and solutions are discussed below throughout the specification, and the scope of the claims should not be limited to addressing only challenges associated with posture monitoring or sensors measuring physical changes in the spine.

### SUMMARY

Described in this disclosure are device, systems, and methods for diagnosing pathologic posture and changing this posture back to optimal posture thereby treating conditions such as low back pain and scoliosis. More specifically, described herein are undergarments with sensors that are used to monitor and measure lower body posture, determine pathologic posture from said measurements, and help alleviate such conditions.

Other systems, methods, features and/or advantages will become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1A illustrates an underwear section alone comprising one or more sensors.

FIG. 1B illustrates a chest harness section comprising one or more sensors, that can be used alone or in combination with the underwear section shown in FIG. 1A.

FIG. 1C illustrates an exemplary system for monitoring posture using the sensors and underwear section of FIG. 1A and/or the sensors and chest harness section of FIG. 1B.

FIG. 2 illustrates an exemplary cloud computing architecture.

FIG. 3 illustrates an exemplary computer for use with the disclosed embodiments.

### DETAILED DESCRIPTION

The present disclosure relates to techniques for positioning one or more sensors on or in undergarments of an individual such that they can be worn to detect posture and/or changes in posture, which can be used to diagnose and/or treat conditions of the musculoskeletal system of the individual.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

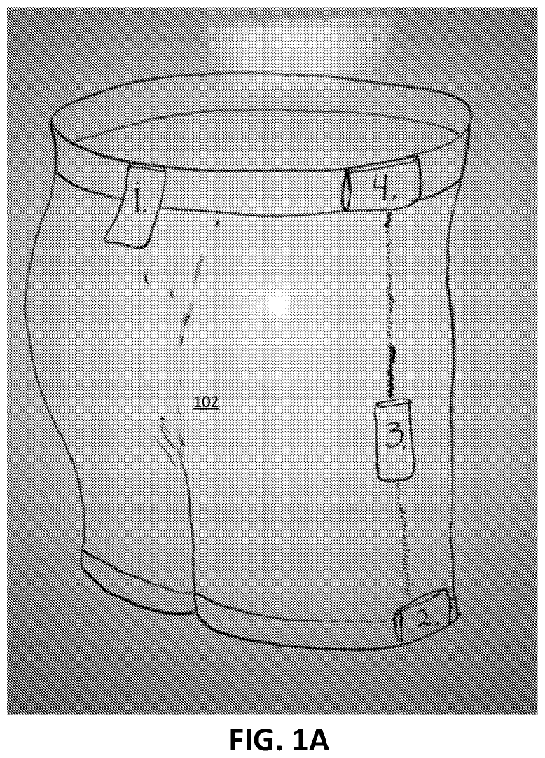

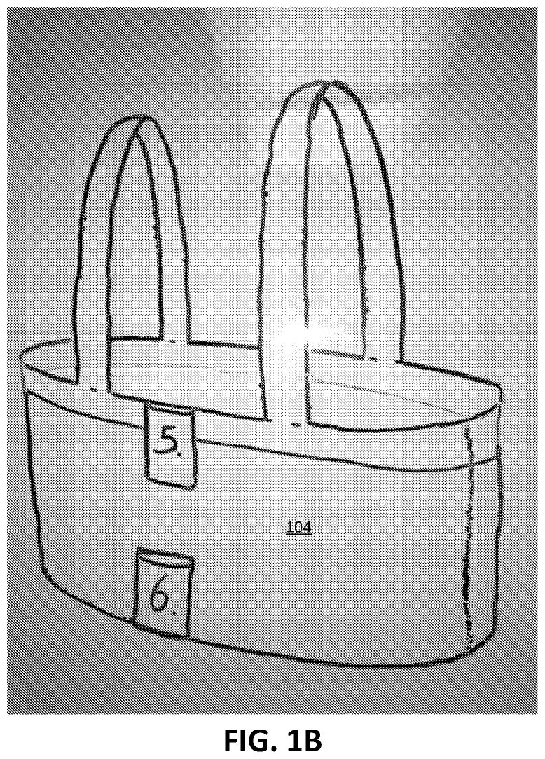

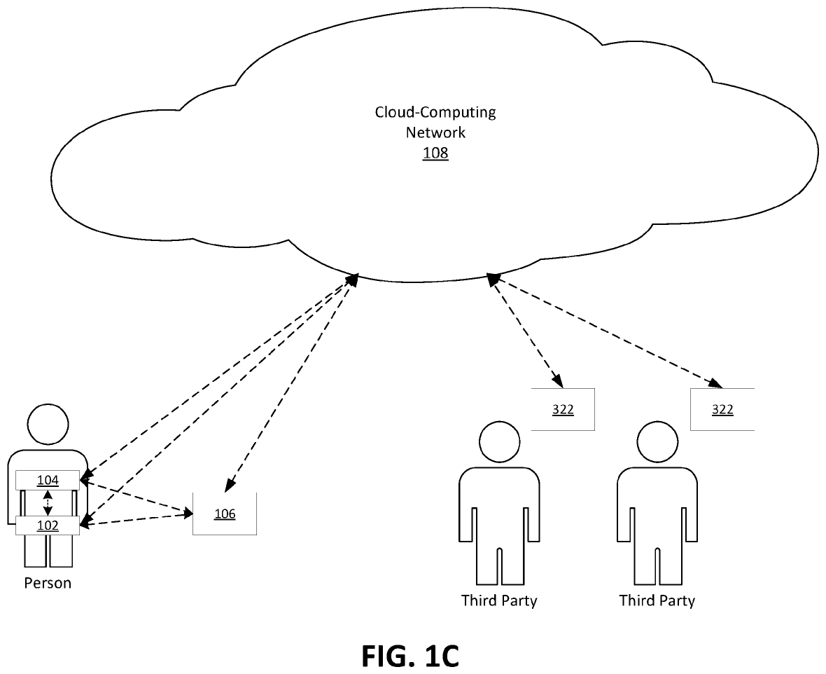

FIG. 1A illustrates an exemplary underwear section alone comprising one or more sensors for monitoring posture of a person and obtaining data associated with such monitoring. Such data can be defined as having multiple categories, with each category having one or more levels of sensitivity. The disclosed system of FIG. 1A can be used for obtaining, storing and distributing data, wherein data in different categories or having different levels of sensitivity can be treated differently within the system. For example, data that identifies or can be used to identify a patient should not be reproduced to all third-parties, as required by laws such as the Health Insurance Portability and Accountability Act of 1996 (HIPAA).

With reference to FIG. 1A, one or more sensors 1, 2, 3, 4, are either attached to, embedded in or otherwise contained and/or held in place by the underwear section 102. The underwear section lays directly on the skin of the person and goes around the pelvis. Each of the one or more sensors 1, 2, 3, 4 comprises one or more of an angular rate sensor, an accelerometer, a gyroscope, a magnetometer, a GPS sensor, a barometer, a sensor that senses physiological aspects of the person such as a blood-pressure sensor, a pulse/heart rate sensor, a blood-oxygen level sensor, a temperature sensor, and the like, and/or combinations thereof.

Though the exemplary illustration of FIG. 1A shows four sensors 1, 2, 3, 4, it is to be appreciated that more or fewer sensors may be used in various embodiments. Generally, each of the one or more sensors 1, 2, 3, 4 comprise a sensing element, a power source (most typically a battery), and a transmitter for transmitting data (for example, Bluetooth technology). Typically, the data is transmitted wirelessly to some other device such as a smartphone, router, computer, tablet, base station or the like, but it is contemplated that in some instances or applications, a wired connection may be used to obtain data from the one or more sensors 1, 2, 3, 4. In some instances, more than one sensor may share a power source and/or a transmitter. Any wiring between sensors may be embedded in or attached to the underwear section 102. In some instances, the one or more sensors 1, 2, 3, 4 are contained within a pocket sewn into the underwear section 102. The pocket may have an opening to facilitate inserting and/or removing the one or more sensors 1, 2, 3, 4. In this manner, the one or more sensors 1, 2, 3, 4, can be removed from the underwear section 102 so that the underwear section 102 can be washed In some instances, the pocket may have a closing mechanism such as a button, zipper, hook and loop fabric (e.g., Velcro™), and the like.

FIG. 1B illustrates an exemplary chest harness section 104 also comprising also one or more sensors 5, 6, that can be used alone or in combination with the underwear section 102 shown in FIG. 1A, as with the above, each of the one or more sensors 5, 6 comprises one or more of an angular rate sensor, an accelerometer, a gyroscope, a magnetometer, a GPS sensor, a barometer, a sensor that senses physiological aspects of the person such as a blood-pressure sensor, a pulse/heart rate sensor, a blood-oxygen level sensor, a temperature sensor, and the like, and/or combinations thereof.

Though the exemplary illustration of FIG. 1B shows two sensors 5, 6, it is to be appreciated that more or fewer sensors may be used in various embodiments. Generally, each of the one or more sensors 5, 6 comprises a sensing element, a power source (most typically a battery), and a transmitter for transmitting data (for example. Bluetooth technology). Typically, the data is transmitted wirelessly to some other device such as a smartphone, router, computer, tablet, base station or the like, but it is contemplated that in some instances or applications, a wired connection may be used to obtain data from the one or more sensors 5, 6. In some instances, more than one sensor may share a power source and/or a transmitter. Any wiring between the one or more sensors 5, 6 may be embedded in or attached to the chest harness section 104. In some instances, the one or more sensors 5, 6 are contained within a pocket sewn into the chest harness section 104. The pocket may have an opening to facilitate inserting and/or removing the one or more sensors 5, 6. In this manner, the one or more sensors 5, 6, can be removed from the chest harness section 104 so that the chest harness section 104 can be washed. In some instances, the pocket may have a closing mechanism such as a button, zipper, hook and loop fabric (e.g., Velcro™), and the like.

In some instances, there may be communications between the one or more sensors 1, 2, 3, 4 of the underwear section 102 and the one or more sensors 5, 6 of the chest harness section 104. Typically, any such communications are wireless communications (e.g., Bluetooth), but in some instances may be wired.

FIG. 1C illustrates an exemplary system for monitoring posture using the one or more sensors 1, 2, 3, 4 and underwear section 102 of FIG. 1A and/or the one or more sensors 5, 6 and chest harness section of FIG. 1B. As shown in FIG. 1C, in some instances, information obtained by the one or more sensors 1, 2, 3, 4, 5, 6 of either of or both the underwear section 102 and the chest harness section 104 is transferred to an intermediate device 106 using a communications interface. This transfer may occur through wires (including fiber optics) and/or be transferred wirelessly. The intermediate device 106 can be, for example, dedicated receivers associated with the one or more sensors 1, 2, 3, 4, 5, 6, smart phones, smart watches, personal computers, tablets, and a variety of other computing devices. Although not illustrated, sensors 1, 2, 3, 4, 5, 6 may include nonvolatile memory for storing historical data, a processor, a battery, and a wireless transmitter. The wireless transmitter can provide any type of wireless communications, including a Bluetooth connection, Wi-Fi connection, RF connection, and others, with the intermediate device 106 and other computing devices. The wireless communications occur, in some embodiments, between paired, authenticated devices, and use encryption and other cryptographic techniques to ensure that communications remain confidential. In other instances, the sensors 1, 2, 3, 4, 5, 6 may include a wireless transmitter capable of communicating directly with a network without use of an intermediate device. For example, the sensors 1, 2, 3, 4, 5, 6 may be connected with a Wi-Fi transceiver that can communicate wirelessly with a LAN, WAN, or other type of network. Alternately or optionally, the sensors 1, 2, 3, 4, 5, 6 may be connected to a wireless transceiver that enable communications directly with a network such as a cloud computing network.

While illustrated as a single unit, portions of the intermediate device 106 may be removable from remaining portions of the intermediate device 106. For example, reusable electronics portions of the intermediate device 106 (e.g., transmitter, battery, memory) may be removable from single use portions of the intermediate device 106 (e.g. and reused with a new single use portion). Further, the intermediate device 106 can include other components to facilitate data communications. For example, the intermediate device 106 may include wired ports, such as a USB port, Ethernet port, and others, for communicating with other devices and providing data.

The one or more sensors 1, 2, 3, 4, 5, 6 of FIGS. 1A and 1B can obtain samples at real-time and/or at predetermined intervals, such as every few seconds, every thirty seconds, every minute, every five minutes, or on demand in response to the occurrence of an event (e.g., a command from a user, detection of a user action, such as user movement, and the like). The wireless transmitters of the one or more sensors 1, 2, 3, 4, 5, 6 may in some instances be turned off or put into a low power state to conserve battery life while one or more measurements are taken over a period of time, and then wake the transmitter back up to wirelessly transmit the one or more measurements to the intermediate device 106 in a batch transfer.

The data transmitted between the one or more sensors 1, 2, 3, 4, 5, 6 and the intermediate device 106 can be any type of data relating to monitoring a person and, in particular to the monitoring of a posture of a person. Transmitted data may also include operation information of the one or more sensors 1, 2, 3, 4, 5, 6, the wireless transmitter or transceiver, battery life, and the like. For example, the one or more sensors 1, 2, 3, 4, 5, 6 may exchange calibration data with respective intermediate device 106 on initial startup and periodically to maintain accuracy of the measurements.

Other examples of data exchanged may include an amount of current or voltage (e.g., raw values) measured by a sensor 1, 2, 3, 4, 5, 6, a timestamp associated with the time when each measurement or value was sampled, alerts related to set values exceeding/falling below predetermined thresholds, detected faults in the system, firmware version, hardware version for the sensor 1, 2, 3, 4, 5, 6 and transmitter, calibration status, the time the senor was started and/or stopped, battery voltage, encryption information, a transmitter identifier number, and the like.

In some instances, the intermediate device 106 may be omitted, and all or a portion of the data may be transmitted directly from some or all of the one or more sensors 1, 2, 3, 4, 5, 6 directly to a network such as the cloud computing architecture 108 using wireless communications technology. For example, the sensors 1, 2, 3, 4, 5, 6 may connected with a cellular chip that uses a modem to transfer data. Or, as described herein, the sensors 1, 2, 3, 4, 5, 6 may be connected with a wireless transceiver such as a Wi-Fi transceiver, a Bluetooth transceiver, and the like. In some instances, there may be at least two data streams—one that is transmitted directly from a sensor 1, 2, 3, 4, 5, 6 to the cloud computing architecture 108 and that which is transmitted to the intermediate device 106 and then from the intermediate device 106 to the cloud computing architecture 108. Any data of any type transmitted between the one or more sensors 1, 2, 3, 4, 5, 6 and the intermediate device 106, or between the intermediate device 106 and the distributed cloud computing architecture 108 or between any one the one or more sensors 1, 2, 3, 4, 5, 6 and the intermediate device 106 and any other physiological monitoring device or any other system, device or person can be considered a data point.

Intermediate device 106 may be a device dedicated to use with the one or more sensors 1, 2, 3, 4, 5, 6, or it may be a device having multiple uses. The combination of the one or more sensors 1, 2, 3, 4, 5, 6 and an intermediate device 106 can, in one embodiment, be an approved medical device, such as a Class III medical device.

Intermediate device 106 may include a processor for performing calculations based on received measurements, memory for storing information, ports for wired communications, and wireless communication circuits, such as Bluetooth, Wi-Fi, or RF circuits. Intermediate device 106 may also be associated with a personal computer, tablet, smart phone, and the like that executes applications. As a result, intermediate device 106 may include hardware components typically associated with personal computing devices, including processor(s), memory, wireless connections, a USB port, and others.

Intermediate device 106 can be a dedicated device or a general-purpose computing device, such as, for example, a smart phone. The smart phone can execute applications dedicated for use with the one or more sensors 1, 2, 3, 4, 5, 6 and other applications. The dedicated application controls the distribution of medical data received from the one or more sensors 1, 2, 3, 4, 5, 6 to other applications executing on the intermediate device 106 to preserve confidentiality and/or user preferences. The dedicated application can also be connected to and provide information to other third-party applications.

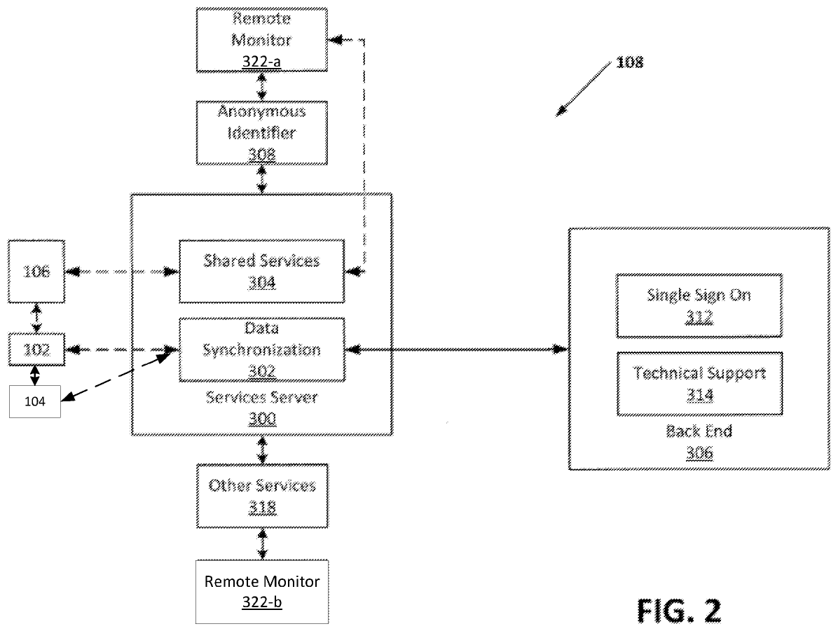

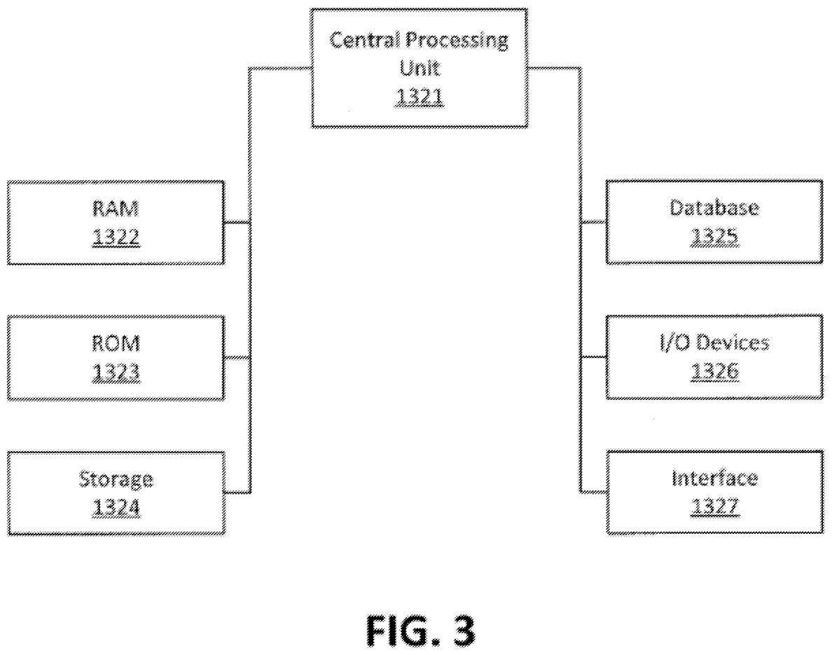

The intermediate device 106 and/or the one or more sensors 1, 2, 3, 4, 5, 6 can transmit data to the distributed cloud computing architecture 108. The distributed cloud computing architecture 108 organizes, stores, analyzes, and provides access to the data by other computers, applications, and third-parties. The distributed cloud computing architecture 108 includes plurality of different servers, storage systems, and software applications executing both locally and across distributed networks. FIGS. 2 and 3 provides more detailed description of distributed cloud computing architecture 108.

Communications within the system can be subject to a number of security protocols. For example, communications can be encrypted and secured, such as HTTPS and SSL communications. The cloud computing architecture 108 may include a firewall that only allows specific and secure communication on defined ports. In addition, the system can use authenticated sessions with a login with name and password for web service methods that a user or remote monitor (described herein) would use to gain access to read or alter their information. The login names and passwords are stored in a secure fashion using hashing and encryption, and patient data including all data posts from the displays can be likewise encrypted and stored in a secure fashion by the cloud computing architecture 108.

Another security measure includes using an authenticated session that times out after a short period of inactivity and also can have a maximum length. Servers can keep an audit trail or history log of all access to the system and all changes made to the system. In addition, third parties accessing data stored by the cloud computing architecture can be required to authenticate themselves and may also be further restricted to only access patients they already know. That is, a user's privilege may require them to already know the patient's internal identifier with the system which would have already been provided by a patient initiated exchange of any identifying information with that consumer.

All of the data can be stored separately in data streams on both or either of the intermediate device 106 and the cloud computing architecture 108. This allows for an audit trail to determine what data came from which device and when. The cloud computing architecture 108 may separately store data received from each intermediate device 106 or data specific to a patient. The data can be stored using metadata by providing a timestamp at which time the data was received at or posted to the cloud computing architecture 108. Accordingly, the cloud computing architecture 108 can track the time at which the last post was received from a particular intermediate device 106 and/or sensor 1, 2, 3, 4, 5, 6. The post might contain new data or data that was previously sent, dropped in transmission due to an error or other system malfunction, and then retransmitting. Metadata allows the intermediate device 106 and/or sensor 1, 2, 3, 4, 5, 6 and the cloud computing architecture 108 to track the last attempted message transmission from the intermediate device 106 and/or sensor 1, 2, 3, 4, 5, 6 and received message transmission by the cloud computing architecture 108. The servers therefore need not examine the actual data that was transmitted but instead rely on the metadata to efficiently store and subsequently retrieve information.

When new data records are created in the system, multiple other computers, devices and services, having proper permissions or authorizations, can be alerted about this data by requesting notifications from the cloud computing architecture. For example, a remote monitor 322 can receive information about the patient by requesting notification of posture information for a specific patient through the cloud computing architecture 108. A remote monitor 322 may comprise, for example, a smart phone, a specialized monitoring device, a smart watch or other personalized wearable electronics, a computer (desktop, laptop, tablet, etc.), and the like. These third-party applications can therefore obtain public information, including the posture information, or other information that they have been provided authorization to receive, whereas a technical support team can also access proprietary private data.

FIG. 2 illustrates an exemplary cloud computing architecture 108. There are a number of challenges associated with receiving and storing large volumes of data. One such challenge is simply the volume of data. Receiving data from intermediate device 106 and/or sensor 1, 2, 3, 4, 5, 6 in real-time or on a periodic basis, such as every five minutes, presents a large load on servers to store the data. This may be compounded by thousands of additional displays associated with other patients all transmitting data to the same server. The cloud computing architecture 108 can both store long-term data that can be used by third-parties, technical support, and other systems, and provide fast access for recent data from a large number of patients. In addition, security issues arise for receiving the data and storing it in a secure fashion, and ensuring that only authorized devices obtain access to the data. Further, some data may be sent through a display but it may be desired that the display not be able to access it. An example is system diagnostic information sent from a transmitter to a server via a phone that can be used by technical support but is proprietary and should not be displayed to a user. The system of FIG. 2 allows different data to be treated differently, with varying levels of access by different system components.

In FIG. 2, intermediate device 106 and/or sensor 1, 2, 3, 4, 5, 6 transmit data to services server 300. The services server 300 provides the functions for coordinating storage, retrieval, and notifications relating to posture information in the system. In one embodiment, the intermediate device 106 and/or sensor 1, 2, 3, 4, 5, 6 transmit data to the services server 300 using, for example, HTTPS web services. The data includes, for example, position information such as lumbar lordosis, pelvic tilt or how much time the subject spends in a flexed posture and other types of information such as exercising information or other health-related information. The intermediate device 106 and/or sensor 1, 2, 3, 4, 5, 6 send the data to the services server 300 automatically in one embodiment. The data includes data from the one or more sensors 1, 2, 3, 4, 5, 6 as well as any additional data added by the intermediate device 106.

Real-time data can be provided, for example, as it occurs (real-time or near real-time) and/or periodically (e.g., every five minutes) from intermediate device 106 and/or sensor 1, 2, 3, 4, 5, 6. Bulk data can be provided, for example, in real-time or periodically such as once every hour from intermediate device 106 and/or sensor 1, 2, 3, 4, 5, 6. Bulk data includes internal system data, such as system operation data, that typically would not be provided to any third-parties. The real-time data and bulk data points can be different or overlapping. For example, bulk data can also include posture information that are also real-time data values. The data can be sent directly from an intermediate device, such as a smart phone, or from the intermediate device 106 and/or sensor 1, 2, 3, 4, 5, 6 to a personal computer or other computing device that uploads the data to the services server 300. For example, the intermediate device 106 can be a personal computer, and the personal computer uploads data through a wired or wireless link. In another embodiment, intermediate device 106 is a smart phone and it uploads data using an application. The real-time and bulk data can be synchronized with the services server 300 in different manners, such as at different time intervals, to facilitate separate storage and retrieval of real-time and bulk data by the cloud computing architecture 108.

In one embodiment, the transmitter of the intermediate device 106 and/or sensor 1, 2, 3, 4, 5, 6 can encrypt all or a portion of the bulk data and pass it through the intermediate device 106 and/or sensor 1, 2, 3, 4, 5, 6 to a services server 300 (see FIG. 2) using a key stored on the transmitter. The transmitter can also encrypt all or a portion of the real-time data using, for example, Bluetooth encryption or other techniques, and the intermediate device 106 can receive the real-time data, decrypt some or all of it for use and display, and forward the real-time data to the services server 300 for storage.

Referring to FIG. 2, services server(s) 300 stores data for a predetermined amount of time, such as thirty days, and synchronizes data to other devices, applications, and outside companies, along with the back-end 306. The services server 300 and back-end 306 can employ different levels of security for different types of data. The services server(s) 30) includes shared services server 304. Shared services server(s) 304 store the real-time data separately from the bulk data. The displays can send the data separately or together, and the data can be separated into real-time and bulk data by the intermediate device 106 and/or sensor 1, 2, 3, 4, 5, 6, or the services servers. In one embodiment, the shared services server 304 stores data for only a predetermined amount of time. This allows fast searching and access to shared data, and also limits the amount of data stored on shared services server 304. For example, shared services server 304 only stores the data for past 30 days, allowing data to be stored for only as long as other devices would need to retrieve the data. In other aspects, the shared services server 300 can store data for time periods greater than or less than 30 days.

The services server 300 supports gathering the data posts on a patient-by-patient, and stream-by-stream basis. A client, such as an intermediate device 106 and/or sensor 1, 2, 3, 4, 5, 6, other service 318, remote monitor device 322-a, 322-b, or other system component can subsequently request data by asking for a specific range of data for each patient. The range of data can be based on the time the data was posted to the server. In one embodiment, each transmission of data by a display can be assigned to a posting identifier. A request can be made to obtain all data posts that came after a posting identifier that can also be tracked by the client.

The system can maintain separate record “streams” of posted information for each patients source display, such as a smart phone and a receiver dedicated to use with the intermediate device 106 and/or sensor 1, 2, 3, 4, 5, 6. Each post can identify the source type by indicating which display posted the data. This may lead to duplicate posting of patient data, from multiple sources. The services server 300, in one embodiment, separately stores these streams of data posts to reduce the complexity on the posting display devices by allowing the display devices to create incremental posts relative only to their own self-contained contiguous data. Consumers may then maintain or report on the differences between the streams or may combine the contents of the streams as desired/required.

Examples of other devices that would access recent data through shared services server 304 include remote monitors 322-a, 322-b that receive data, alerts, information, and the like in real-time. A remote monitor 322-a, 322-b can be under the control of a person who monitors the posture of another patient. For example, one or more of an insurance provider, a physician, a therapist, a family-member of the person, or anyone else designated by the person can monitor posture of a person using a remote monitor 322-a, 322-b.

One challenge that may arise with remote monitors 322-a, 322-b is that storing any identifying information for the remote monitor could place those interactions under government privacy laws and regulation such as, for example, HIPAA regulations. It would be preferable to avoid storing non-patient (i.e., remote monitor 322-a, 322-b) information in the cloud computing architecture to avoid implicating any privacy law or regulation. Accordingly, in one embodiment the cloud computing architecture 108 need not receive or store any of a remote monitor's personal information. Instead, in one embodiment the remote monitor 322-a can be assigned a digital signature or other secure anonymous identifier 308 that is associated with the remote monitor 322-a, but the relationship is not stored in the cloud computing architecture. For example, the registration process for a remote monitor 322-a can result in the generation of a unique number that is an anonymous identification of the follower. Communications within the system, such as between the shared services server 304 and a remote monitor device 322-a may use the anonymous identifier 308 instead of information that would identify the remote monitor 322-a.

The cloud computing architecture 108 may also include back-end server(s) 306. The back-end 306 receives real-time data from shared services servers 304 and bulk data from data synchronization server 302. Back-end 306 stores historical data over a certain age (e.g., thirty days old) and receives requests for access to data through other services 318 that is more than thirty days old.

The back-end 306 functions as a data warehouse that can store data either permanently or for longer periods of time for archival purposes. Technical support unit 314 provides technical support to users and patients for any issues with system operation. Technical support unit 314 receives posture data and other real-time and bulk data and can permanently store the data to assist with future technical support issues. For example, a patient establishes alerts on intermediate device 106 and/or sensors 1, 2, 3, 4, 5, 6 for when posture thresholds cross a defined level or experience a defined rate of change.

Single sign on server 312 provides a single sign-on for patients and users accessing a number of different applications and the system. If the system were comprised of separate systems, applications, and components, the user experience may not be seamless, as the user would need to log into separate systems. Accordingly, the smart phones and other displays can log into the system through the cloud infrastructure 108 using single sign on server 312. In one example, a transmitter identifier can be printed on an intermediate device 106 and used as the sign on to correlate a transmitter with a particular patient. In addition, users can have a login name and password, and a variety of different encryption algorithms can be used in the authentication process.

Other services 318 can include a number of other services that seek access to patient data. As an example, a medical professional (e.g., doctor) can request access through other services 318 to patient data stored by services server. The other services 318, in one embodiment, receive real-time data through services server 300 for the past thirty days. Other services 318 can synchronize data and save data periodically through services servers 300. For example, some other applications can request data hourly, others daily, and others weekly to have the data from services servers 300. For example, other services 318 can include applications that request data to perform data analytics, both for individual patients and for classes of patients. When other services 318 request data beyond the age range stored by services servers 300, that request is sent to and processed by back-end 306, which stores longer-term archived bulk and real-time data. The timing as to when various components of the system can request access to bulk and real-time data can vary. For example, the cloud computing architecture can restrict other services 318 to only accessing data once per day, allowing full access at any time, or on a variety of other timeframes.

It will be appreciated that the cloud computing architecture 108 of FIG. 2 can include fewer or additional components. In addition, the system can include a plurality of cloud computing architectures so that fewer than all of the displays transmit data to a single cloud computing architecture. For example, a plurality of connected cloud computing architectures can be used throughout different geographical regions, although other arrangements are also possible to distribute the computing load.

The above-described devices, systems and method may be used for treating low back pain and scoliosis by retraining posture to a more lordotic or anteriorly tilted pelvic position. This can be done through biofeedback whereby the described device vibrates or beeps when the prescribed posture is lost. The device may also educate both the patient and healthcare provider regarding whether or not prescribed postural change is being complied with. As provided herein, one or more sensors 1, 2, 3, 4, 5, 6 located in one or both of an underwear section 102 and a chest harness section 104. The relationship between these electrical sensor “units” defines a subject's posture. Some examples are the angular difference between sensor 1 and 6 defines a traditional lumbar lordosis angle. Also, the difference between sensors 2 or 3 and sensors 1 or 4 define the pelvic tilt. In some instances, the one or more sensors 1, 2, 3, 4, 5, 6 are calibrated. This may involve registering the angular relationship between the units on the subject while the subject is laying prone on a flat surface or while obtaining a lateral x-ray so that exact radiographic parameters can be measured and calibrated to the angular relationship between the units. As disclosed herein, devices, systems and method may be used to record the subject's posture during the day and alerting the person and/or others to pathologic positions and also good postures. The subject's postures can be recorded and analyzed through a separate recorder or through a smartphone via its Bluetooth or wireless data transmission capabilities. The subject and others (e.g., parents, healthcare provider, etc.) can monitor and diagnose pathologic posture as well as the results of treatment interventions toward better postures.

FIG. 3 illustrates an exemplary computer. Sensors 1, 2, 3, 4, 5, 6, intermediate device 106, the cloud computing architecture 108 and associated servers, as well as other system components, can include all or some of the components shown in FIG. 3.

The computers may include one or more hardware components such as, for example, a central processing unit (CPU) 1321, a random-access memory (RAM) module 1322, a read-only memory (ROM) module 1323, a storage 1324, a database 1325, one or more input/output (I/O) devices 1326, and an interface 1327. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 1324 may include a software partition associated with one or more other hardware components. It is understood that the components listed above are exemplary only and not intended to be limiting.

CPU 1321 may include one or more processors, each configured to execute instructions and process data to perform one or more functions associated with a computer for monitoring posture. CPU 1321 may be communicatively coupled to RAM 1322, ROM 1323, storage 1324, database 1325, I/O devices 1326, and interface 1327. CPU 1321 may be configured to execute sequences of computer program instructions to perform various processes. The computer program instructions may be loaded into RAM 1322 for execution by CPU 1321.

RAM 1322 and ROM 1323 may each include one or more devices for storing information associated with operation of CPU 1321. For example, ROM 1323 may include a memory device configured to access and store information associated with controller 1220, including information for identifying, initializing, and monitoring the operation of one or more components and subsystems. RAM 1322 may include a memory device for storing data associated with one or more operations of CPU 1321. For example, ROM 1323 may load instructions into RAM 1322 for execution by CPU 1321.

Storage 1324 may include any type of mass storage device configured to store information that CPU 1321 may need to perform processes consistent with the disclosed embodiments. For example, storage 1324 may include one or more magnetic and/or optical disk devices, such as hard drives, CD-ROMs, DVD-ROMs, or any other type of mass media device.

Database 1325 may include one or more software and/or hardware components that cooperate to store, organize, sort, filter, and/or arrange data used by CPU 1321. For example, database 1325 may data relating to monitoring posture, associated metadata, and health information. It is contemplated that database 1325 may store additional and/or different information than that listed above.

V/O devices 1326 may include one or more components configured to communicate information with a user associated with the device shown in FIG. 3. For example, I/O devices 1326 may include a console with an integrated keyboard and mouse to allow a user to maintain a database of images, update associations, and access digital content. I/O devices 1326 may also include a display including a graphical user interface (GUI) for outputting information on a monitor. I/O devices 1326 may also include peripheral devices such as, for example, a printer for printing information associated with controller 1220, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1327 may include one or more components configured to transmit and receive data via a communication network, such as the Internet, a local area network, a workstation peer-to-peer network, a direct link network, a wireless network, or any other suitable communication platform. For example, interface 1327 may include one or more modulators, demodulators, multiplexers, demultiplexers, network communication devices, wireless devices, antennas, modems, and any other type of device configured to enable data communication via a communication network.

Any combination of one or more computer readable medium(s) may be utilized. The computer readable medium may be a computer readable signal medium or a computer readable storage medium. A computer readable storage medium may be, for example, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. Program code embodied on a computer readable medium may be transmitted using any appropriate medium, including but not limited to wireless, wireline, optical fiber cable, RF, etc., or any suitable combination of the foregoing.

Computer program code for may be written in any combination of one or more programming languages, including an object-oriented programming language such as Java, Smalltalk, C++, or the like, and conventional procedural programming languages, such as the “C” programming language or similar programming languages. The program code may execute entirely on the computing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

While this specification contains many specific implementation details, these should not be construed as limitations on the claims. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

It should be appreciated that the logical operations described herein with respect to the various figures may be implemented (1) as a sequence of computer implemented acts or program modules (i.e., software) running on a computing device, (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein. It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. An article of manufacture comprising:
a piece of clothing that lays directly on the skin, said piece of clothing comprising at least a first piece that goes around the pelvis comprising an underwear section;
one or more sensors, wherein each of the one or more sensors are embedded in, contained by, or otherwise attached to the underwear section,
wherein data obtained by the one or more sensors determines a posture of a person wearing the underwear section.

2. The article of manufacture of claim 1, wherein each of the one or more sensors of the underwear section comprise a sensor element, a power source and a transmitter.

3. The article of manufacture of claim 1, wherein two or more of the one or more sensors of the underwear section share a power source and/or a transmitter, said two or more sensors connected by wiring.

4. The article of manufacture of any one of claims 1-3, wherein a first one of the one or more sensors is located in a back or front of the underwear section against either the dorsal sacrum or navel of the person and a second one of the one or more sensors is located either laterally, anteriorly, or posteriorly on a proximal thigh of the person within the underwear section.

5. The article of manufacture of any one of claims 1-4, wherein the piece of clothing comprises a second piece, said second piece comprising a chest harness section, said chest harness section further comprising one or more sensors, wherein each of the one or more sensors are embedded in, contained by, or otherwise attached to the chest harness sections,
wherein a relationship between data obtained by the one or more sensors of the underwear section and the one or more sensors of the chest harness section determines the posture of the person wearing the underwear section and the chest harness section.

6. The article of manufacture of claim 5, wherein each of the one or more sensors of the chest harness section comprise a sensor element, a power source and a transmitter.

7. The article of manufacture of claim 5, wherein two or more of the one or more sensors of the chest harness section share a power source and/or a transmitter, said two or more sensors connected by wiring.

8. The article of manufacture of any one of claims 1-7, wherein the one or more sensors of the underwear section and/or the one or more sensors of the chest harness section communicate wirelessly with one another and/or with an intermediate device.

9. The article of manufacture of claim 8, wherein the intermediate device comprises a dedicated receiver associated with the one or more sensors of the underwear section and/or the one or more sensors of the chest harness section, a smart phone, a smart watch or other person wearable electronics, a personal computer, including a laptop computer, a tablet computing device, a router, and/or a variety of other computing devices.

10. The article of manufacture of claim 9, wherein the intermediate device can transmit and/or receive data from a cloud computing network.

11. The article of manufacture of any one of claims 1-10, wherein the article of manufacture is in the medical field to treat low back pain and scoliosis.

12. The article of manufacture of claim 11, wherein an x-ray is obtained while the person wears the article of manufacture so that the person's radiographic parameters can be quantified and a location of the person's lumbar lordosis or pelvic tilt can be estimated during activities of daily living for placement of the one or more sensors of the underwear section and/or the one or more sensors of the chest harness section.

13. The article of manufacture of any one of claim 11 or 12, wherein the one or more sensors of the underwear section and/or the one or more sensors of the chest harness section are calibrated while the person lays prone on a flat surface, which creates a reproducible calibration point which will not change with posture.

14. The article of manufacture of any one of claims 11-13, wherein data from the one or more sensors of the underwear section and/or the one or more sensors of the chest harness section are used to inform both pathologic and optimal posture notification of either the person or other interested parties and/or to provide biofeedback.

15. The article of manufacture of claim 14, wherein data from the one or more sensors of the underwear section and/or the one or more sensors of the chest harness section are used to define optimal posture based on human population demographic factors (example: genotype, phenotype, pelvic incidence, age) and then which notifies the person or the other interested parties as to the person's variation from this population norm based on analysis of the data.

16. The article of manufacture of any one of claims 1-15, wherein the one or more sensors of the underwear section and/or the one or more sensors of the chest harness section comprise one or more of an angular rate sensor, an accelerometer, a gyroscope, a magnetometer, a GPS sensor, a barometer, a sensor that senses physiological aspects of the person such as a blood-pressure sensor, a pulse/heart rate sensor, a blood-oxygen level sensor, a temperature sensor, and the like, and/or combinations thereof.

17. The article of manufacture of claim 16, wherein data from the one or more sensors of the underwear section and/or the one or more sensors of the chest harness section are used to quantify kinematics and kinetics of the person.

18. A method of monitoring the posture of a person using an article of manufacture.

19. The method of claim 18, wherein the article of manufacture comprises:
a piece of clothing that lays directly on the skin, said piece of clothing comprising at least a first piece that goes around the pelvis comprising an underwear section;
one or more sensors, wherein each of the one or more sensors are embedded in, contained by, or otherwise attached to the underwear section,
wherein data obtained by the one or more sensors determines a posture of a person wearing the underwear section.

20. The method of claim 19, wherein each of the one or more sensors of the underwear section comprise a sensor element, a power source and a transmitter.

21. The method of claim 19, wherein two or more of the one or more sensors of the underwear section share a power source and/or a transmitter, said two or more sensors connected by wiring.

22. The method of any one of claims 19-21, wherein a first one of the one or more sensors is located in a back or front of the underwear section against either the dorsal sacrum or navel of the person and a second one of the one or more sensors is located either laterally, anteriorly, or posteriorly on a proximal thigh of the person within the underwear section.

23. The method of any one of claims 19-22, wherein the piece of clothing comprises a second piece, said second piece comprising a chest harness section, said chest harness section further comprising one or more sensors, wherein each of the one or more sensors are embedded in, contained by, or otherwise attached to the chest harness sections,
wherein a relationship between data obtained by the one or more sensors of the underwear section and the one or more sensors of the chest harness section determines the posture of the person wearing the underwear section and the chest harness section.

24. The method of claim 23, wherein each of the one or more sensors of the chest harness section comprise a sensor element, a power source and a transmitter.

25. The method of claim 23, wherein two or more of the one or more sensors of the chest harness section share a power source and/or a transmitter, said two or more sensors connected by wiring.

26. The method of any one of claims 19-25, wherein the one or more sensors of the underwear section and/or the one or more sensors of the chest harness section communicate wirelessly with one another and/or with an intermediate device.

27. The method of claim 26, wherein the intermediate device comprises a dedicated receiver associated with the one or more sensors of the underwear section and/or the one or more sensors of the chest harness section, a smart phone, a smart watch or other person wearable electronics, a personal computer, including a laptop computer, a tablet computing device, a router, and/or a variety of other computing devices.

28. The method of claim 27, wherein the intermediate device can transmit and/or receive data from a cloud computing network.

29. The method of any one of claims 19-28, wherein the article of manufacture is in the medical field to treat low back pain and scoliosis.

30. The method of claim 29, wherein an x-ray is obtained while the person wears the article of manufacture so that the person's radiographic parameters can be quantified and a location of the person's lumbar lordosis or pelvic tilt can be estimated during activities of daily living for placement of the one or more sensors of the underwear section and/or the one or more sensors of the chest harness section.

31. The method of any one of claim 29 or 30, wherein the one or more sensors of the underwear section and/or the one or more sensors of the chest harness section are calibrated while the person lays prone on a flat surface, which creates a reproducible calibration point which will not change with posture.

32. The method of any one of claims 29-31, wherein data from the one or more sensors of the underwear section and/or the one or more sensors of the chest harness section are used to inform both pathologic and optimal posture notification of either the person or other interested parties and/or to provide biofeedback.

33. The method of claim 32, wherein data from the one or more sensors of the underwear section and/or the one or more sensors of the chest harness section are used to define optimal posture based on human population demographic factors (example: genotype, phenotype, pelvic incidence, age) and then which notifies the person or the other interested parties as to the person's variation from this population norm based on analysis of the data.

34. The method of any one of claims 19-33, wherein the one or more sensors of the underwear section and/or the one or more sensors of the chest harness section comprise one or more of an angular rate sensor, an accelerometer, a gyroscope, a magnetometer, a GPS sensor, a barometer, a sensor that senses physiological aspects of the person such as a blood-pressure sensor, a pulse/heart rate sensor, a blood-oxygen level sensor, a temperature sensor, and the like, and/or combinations thereof.

35. The method of claim 34, wherein data from the one or more sensors of the underwear section and/or the one or more sensors of the chest harness section are used to quantify kinematics and kinetics of the person.

36. A system for monitoring the posture of a person comprising:
an article of manufacture;
an intermediate device, wherein the intermediate device communicates with the one or more sensors of the underwear section and/or the one or more sensors of the chest harness section; and
a processor, wherein the processor:
receives data from one or more sensors of the underwear section and/or the one or more sensors of the chest harness section; and
determines a posture of the person using data from one or more sensors of the underwear section and/or the one or more sensors of the chest harness section.

37. The system of claim 36, wherein the article of manufacture comprises:
a piece of clothing that lays directly on the skin, said piece of clothing comprising at least a first piece that goes around the pelvis comprising an underwear section;
one or more sensors, wherein each of the one or more sensors are embedded in, contained by, or otherwise attached to the underwear section,
wherein data obtained by the one or more sensors determines a posture of a person wearing the underwear section.

38. The system of claim 37, wherein each of the one or more sensors of the underwear section comprise a sensor element, a power source and a transmitter.

39. The system of claim 37, wherein two or more of the one or more sensors of the underwear section share a power source and/or a transmitter, said two or more sensors connected by wiring.

40. The system of any one of claims 37-39, wherein a first one of the one or more sensors is located in a back or front of the underwear section against either the dorsal sacrum or navel of the person and a second one of the one or more sensors is located either laterally, anteriorly, or posteriorly on a proximal thigh of the person within the underwear section.

41. The system of any one of claims 37-40, wherein the piece of clothing comprises a second piece, said second piece comprising a chest harness section, said chest harness section further comprising one or more sensors, wherein each of the one or more sensors are embedded in, contained by, or otherwise attached to the chest harness sections,
wherein a relationship between data obtained by the one or more sensors of the underwear section and the one or more sensors of the chest harness section determines the posture of the person wearing the underwear section and the chest harness section.

42. The system of claim 41, wherein each of the one or more sensors of the chest harness section comprise a sensor element, a power source and a transmitter.

43. The system of claim 41, wherein two or more of the one or more sensors of the chest harness section share a power source and/or a transmitter, said two or more sensors connected by wiring.

44. The system of any one of claims 37-43, wherein the one or more sensors of the underwear section and/or the one or more sensors of the chest harness section communicate wirelessly with one another and/or with an intermediate device.

45. The system of claim 44, wherein the intermediate device comprises a dedicated receiver associated with the one or more sensors of the underwear section and/or the one or more sensors of the chest harness section, a smart phone, a smart watch or other person wearable electronics, a personal computer, including a laptop computer, a tablet computing device, a router, and/or a variety of other computing devices.

46. The system of claim 45, wherein the intermediate device can transmit and/or receive data from a cloud computing network.

47. The system of any one of claims 37-46, wherein the article of manufacture is in the medical field to treat low back pain and scoliosis.

48. The system of claim 47, wherein an x-ray is obtained while the person wears the article of manufacture so that the person's radiographic parameters can be quantified and a location of the person's lumbar lordosis or pelvic tilt can be estimated during activities of daily living for placement of the one or more sensors of the underwear section and/or the one or more sensors of the chest harness section.

49. The system of any one of claim 47 or 48, wherein the one or more sensors of the underwear section and/or the one or more sensors of the chest harness section are calibrated while the person lays prone on a flat surface, which creates a reproducible calibration point which will not change with posture.

50. The system of any one of claims 47-49, wherein data from the one or more sensors of the underwear section and/or the one or more sensors of the chest harness section are used to inform both pathologic and optimal posture notification of either the person or other interested parties and/or to provide biofeedback.

51. The system of claim 50, wherein data from the one or more sensors of the underwear section and/or the one or more sensors of the chest harness section are used to define optimal posture based on human population demographic factors (example: genotype, phenotype, pelvic incidence, age) and then which notifies the person or the other interested parties as to the person's variation from this population norm based on analysis of the data.

52. The system of any one of claims 37-51, wherein the one or more sensors of the underwear section and/or the one or more sensors of the chest harness section comprise one or more of an angular rate sensor, an accelerometer, a gyroscope, a magnetometer, a GPS sensor, a barometer, a sensor that senses physiological aspects of the person such as a blood-pressure sensor, a pulse/heart rate sensor, a blood-oxygen level sensor, a temperature sensor, and the like, and/or combinations thereof.

53. The system of claim 52, wherein data from the one or more sensors of the underwear section and/or the one or more sensors of the chest harness section are used to quantify kinematics and kinetics of the person.

54. The system of any one of claims 36-53, wherein the determined posture information about the person is electronically provided to the person or other interested parties.
